(19) United States
(12) Patent Application Publication
(10) Pub. No.: US 2008/0146922 A1
(43) Pub. Date: Jun. 19, 2008

(54) Control of user interfaces and displays for portable ultrasound unit and docking station

(75) Inventors: Robert W. Steins, Santa Clara, CA (US); Glen W. McLaughlin, Saratoga, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Zonare Medical Systems, Inc., Mountain View, CA (US)

(21) Appl. No.: 11/586,212

(22) Filed: Oct. 24, 2006

(51) Int. Cl. A61B 8/00

(52) U.S. Cl. 600/437

(57) ABSTRACT

Embodiments of the present invention provide ways for controlling a plurality of visual displays and a plurality of user interfaces for a portable ultrasound device which can be mounted to different docking stations or carts to provide and enhance different functionalities and features. In one embodiment, a portable ultrasound device comprises a portable housing; a display control module configured to control a plurality of visual displays, at least one of the visual displays being selectively configurable to provide a user interface display on the visual display for user interface control, at least one of the visual displays being selectively configurable to view an ultrasound image; and a plurality of user interfaces, at least one of the plurality of user interfaces being a separate user interface which is not integrally formed with the portable housing.

### CROSS-REFERENCES TO RELATED APPLICATIONS

NOT APPLICABLE

### BACKGROUND OF THE INVENTION

This invention relates generally to ultrasound imaging systems and, more particularly, to control of user interfaces and displays for a portable ultrasound unit which is configured to mate with a docking station.

Ultrasound equipment is used in a variety of medical applications. Small hand-held ultrasound scanners are used for applications in which portability is at a premium. Such scanners, while portable, are not as full-featured as larger equipment. Accordingly, there remains a need for full-sized cart-based ultrasound scanners. Such cart-based ultrasound scanners, which typically weigh hundreds of pounds, have more capabilities than small portable units. These traditional cart-based scanners can be moved between different rooms in a medical establishment, but are not portable.

U.S. Pat. No. 6,980,419, the entire disclosures of which is incorporated herein by reference, discloses a portable ultrasound unit and docking cart. The portable ultrasound unit may be small enough to be carried in the hands of a medical professional or other user. When appropriate, the portable ultrasound unit may be used in conjunction with the docking cart. The docking cart may enhance the capabilities of the portable unit so that the portable unit's functionality rivals or exceeds the capabilities of traditional cart-based ultrasound equipment.

When the portable unit is mounted to the docking cart, the docking cart transforms the portable unit into a cart-based system with enhanced features and functionality such as improved ergonomics, ease of use, a larger display format, external communications connectivity, multiple transducer connections, and increased data processing capabilities. A clinician display and patient display may be provided on the cart. Communications circuitry in the docking cart may be used to support communications between the docking cart's processor and external networks and devices. The docking cart may receive physiological signals such as cardiac signals and may use this information to synchronize ultrasound imaging operations with a patient's physiological condition. Adjustable user interface controls, data handling features, security features, power control functions, and thermal management capabilities may be provided in the docking cart.

### BRIEF SUMMARY OF THE INVENTION

Embodiments of the present invention provide ways for controlling a plurality of visual displays and a plurality of user interfaces for a portable ultrasound device which can be mounted to different docking stations or carts to provide and enhance different functionalities and features.

In accordance with an aspect of the present invention, a portable ultrasound device comprises a portable housing; a display control module configured to control a plurality of visual displays, at least one of the visual displays being selectively configurable to provide a user interface display on the visual display for user interface control, at least one of the visual displays being selectively configurable to view an ultrasound image; and a plurality of user interfaces, at least one of the plurality of user interfaces being a separate user interface which is not integrally formed with the portable housing.

In some embodiments, the display control module is configured to control visual displays having different sizes and/or resolutions. The portable ultrasound device includes a portable ultrasound device visual display. The portable ultrasound device visual display is selectively configurable to provide a user interface display on the portable ultrasound device visual display as a touch screen for user interface control of one or more of the user interfaces and selectively configurable to view an ultrasound image. At least one of the user interfaces is an integral user interface which is integrally formed with the portable housing. A communication module is configured to communicate with an auxiliary medical device for therapeutic application. The communication module is configured to send control signal to and/or receive control signal from the auxiliary medical device. A security mechanism is provided to lock the portable housing to a surface. A user interface allocation module is configured to allocate user interface functionalities among the plurality of user interfaces. A visual display allocation module is configured to allocate visual display functionalities among a plurality of visual displays.

In specific embodiments, a docking station comprises a docking station visual display which is configurable to view an ultrasound image. The portable ultrasound device comprises a transducer port configured to be coupled to a connector of an ultrasound transducer and a portable ultrasound device ultrasound processing circuitry that accepts first ultrasound image data from the transducer port and processes the first ultrasound image data to generate second ultrasound image data. The docking station comprises digital communications circuitry that supports communication between the docking station and the portable ultrasound device. The docking station visual display is configured to display an ultrasound image based on the second ultrasound image data received from the portable ultrasound device through the digital communications circuitry.

In some embodiments, the docking station comprises digital communications circuitry that supports communication between the docking station and the portable ultrasound device. The docking station comprises a docking station ultrasound processing circuitry that processes the second ultrasound image data received by the docking station from the portable ultrasound device through the digital communications circuitry. The docking station visual display is configured to display an ultrasound image based on processed data from the docking station ultrasound processing circuitry.

In specific embodiments, the docking station comprises a docking station user interface to receive user input of display instruction for the docking station visual display. At least one of the user interfaces of the portable ultrasound device receives user input of display instruction for the docking station visual display. The portable ultrasound device comprises a portable ultrasound device visual display, which is selectively configurable to provide a user interface display on the portable ultrasound device visual display, as a touch screen for user interface control to receive user input of display instruction for the docking station visual display.

In accordance with another aspect of the invention, an ultrasound system includes a portable ultrasound unit and a docking cart. The portable ultrasound unit comprises a portable housing, a portable ultrasound unit visual display, and a plurality of user interfaces. The portable ultrasound unit visual display is selectively configurable to provide a user interface display on the visual display for user interface control of one of more of the user interfaces and selectively configurable to view an ultrasound image. The docking cart comprises a docking cart visual display which is configurable to view an ultrasound image. The portable ultrasound unit further comprises a visual display allocation module configured to allocate visual display functionalities between the portable ultrasound unit visual display and the display cart visual display.

In some embodiments, the plurality of user interfaces include at least one of a separate user interface which is not integrally formed with the portable housing; or an integral user interface which is integrally formed with the portable housing. A security mechanism is provided to lock the portable housing of the portable ultrasound unit to the docking cart.

In some embodiments, the portable ultrasound unit comprises a transducer port configured to be coupled to a connector of an ultrasound transducer and a portable ultrasound unit ultrasound processing circuitry that accepts first ultrasound image data from the transducer port and processes the first ultrasound image data to generate second ultrasound image data. The docking cart comprises digital communications circuitry that supports communication between the docking cart and the portable ultrasound unit. The docking cart visual display is configured to display an ultrasound image based on the second ultrasound image data received from the portable ultrasound unit through the digital communications circuitry. In some other embodiments, the docking cart comprises digital communications circuitry that supports communication between the docking cart and the portable ultrasound unit. The docking cart comprises a docking cart ultrasound processing circuitry that processes the second ultrasound image data received by the docking cart from the portable ultrasound unit through the digital communications circuitry. The docking cart visual display is configured to display an ultrasound image based on processed data from the docking cart ultrasound processing circuitry.

In specific embodiments, at least one of the user interfaces of the portable ultrasound unit receives user input of display instruction for the docking cart visual display. The portable ultrasound unit visual display is selectively configurable to provide a user interface display on the portable ultrasound unit visual display, as a touch screen for user interface control to receive user input of display instruction for the docking cart visual display.

In accordance with another aspect of the present invention, a portable ultrasound device comprises a portable housing; a portable device processor; a portable device visual display; and at least one portable device user interface. The portable housing is configured to be mounted to any one of a plurality of docking stations. The portable device processor is configured to interface with a docking station processor of the docking station for allocating user interface functionalities and/or visual display functionalities between the portable ultrasound device and the docking station. The visual display functionalities include displaying an ultrasound image.

In some embodiments, the docking station has a docking station visual display, and wherein the visual display functionalities are allocated between the portable device visual display and the docking station visual display. The portable device visual display may be selectively configurable to provide a user interface display on the portable device visual display, as a touch screen for user interface control to receive user input of display instruction for the docking station visual display. The visual display functionalities may be fully allocated to the docking station visual display.

In specific embodiments, the docking station has at least one docking station user interface, and wherein the user interface functionalities are allocated between the at least one portable device user interface and the at least one docking station user interface. The user interface functionalities may be fully allocated to the at least one docking station user interface.

### BRIEF DESCRIPTION OF THE DRAWINGS

FIG. 1 is a schematic diagram of an illustrative portable ultrasound unit and docking cart.

FIG. 2 is a perspective view of an illustrative portable ultrasound unit with an available ultrasound transducer port.

FIG. 3 is a perspective view of an illustrative portable ultrasound unit with an attached ultrasound transducer.

FIG. 4 is a diagram showing how a portable ultrasound unit may be inserted into a mating receptacle on a docking cart.

FIG. 5 is a diagram showing how a docking cart receptacle for the portable ultrasound unit may be configured so as not to block the transducer port of the portable ultrasound unit.

FIG. 6 is a diagram showing how a docking cart and portable ultrasound unit may have mating electrical connectors.

FIG. 7 is a schematic block diagram of an illustrative portable ultrasound unit in accordance with an embodiment of the present invention.

FIG. 8 is a flow diagram illustrating allocation and control of user interfaces and displays in the portable ultrasound unit according to an embodiment of the present invention.

FIG. 9 is a schematic block diagram of an illustrative docking cart in accordance with an embodiment of the present invention.

FIG. 10 is a perspective view of an illustrative docking cart in accordance with an embodiment of the present invention.

FIG. 11 is a perspective view of an illustrative docking cart in accordance with another embodiment of the present invention.

FIG. 12 shows a touch screen main panel example.

FIG. 13 shows a Doppler mode panel example.

FIG. 14 shows a preset sub-panel example.

FIG. 15 shows a preset touch screen configuration page example.

FIG. 16 is a simplified view of a latch mechanism for locking a portable unit to a surface of a cart.

### DETAILED DESCRIPTION OF THE INVENTION

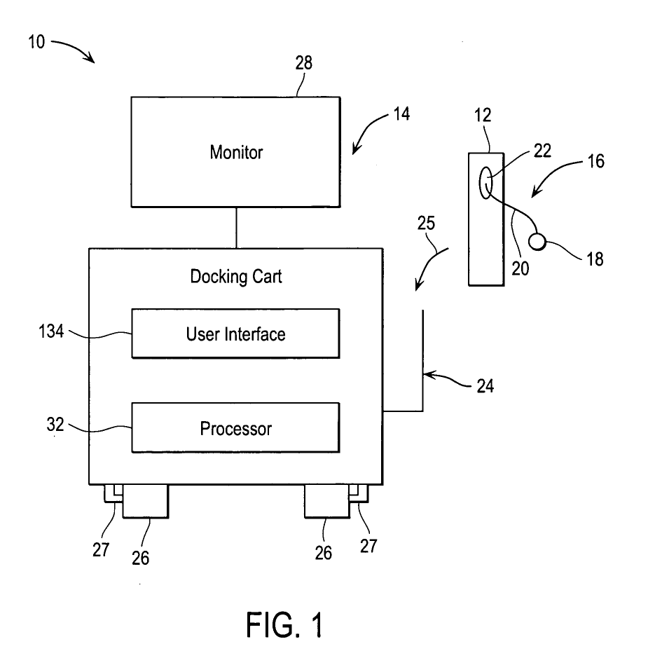

An exemplary embodiment of the present invention is directed to a portable ultrasound unit or device 12 and a docking cart 14 in an ultrasound system 10 as shown in FIG. 1. The portable ultrasound unit 12 may be small and light enough to be easily carried by a user (e.g., a physician or other clinician in a medical setting or other suitable individual). The unit may, if desired, be small and lightweight enough to be carried in a single outstretched arm.

Standard cart-based ultrasound equipment generally weighs hundreds of pounds and is either stationary or is movable only with considerable effort. Accordingly, portable ultrasound units such as the unit 12 may often be much more appropriate to use than traditional cart-based ultrasound systems. For example, the portable ultrasound unit 12 may be used when ultrasound capabilities are needed in the field (e.g., in an ambulance) or even in a clinical setting such as a hospital in which the ability to easily transport the unit 12 from location to location is important. The weight of the portable unit 12 may be on the order of 5 to 10 pounds or less or may be any other suitable weight. The size of the portable unit 12 may be on the order of 4 inches times 10 inches by 12 inches or any other suitable size. These are merely illustrative weights and dimensions. The portable unit 12 may have any suitable size and weight as desired.

Ultrasound images may be gathered using an ultrasound transducer 16. The transducer 16 may have a transducer head 18 and may be connected to the portable unit 12 using a cable 20 and connector 22 or other suitable interconnection arrangement. Different transducers may be used for making different types of ultrasound measurements. For example, medium frequency transducers with phased arrays may be particularly useful for applications in cardiology or abdominal imaging. High-frequency linear transducers may be used for muscular work. Curvilinear transducer heads may be preferred when making obstetrical measurements. Probe-based and catheter-based ultrasonic transducers may also be used with the portable unit 12 if desired. To allow different transducers to be used, the portable ultrasound unit 12 may have a transducer port to which different transducer connectors 22 may be attached, as needed.

The docking cart 14 may have a docking structure 24 that allows the portable unit 12 to be connected to the docking cart 14. The docking structure 24 may be a shelf, receptacle, drawer, slot, recess, clasp, mating protrusion, or any other docking structure that facilitates attachment of the portable unit 12 to the docking cart 14. In the example of FIG. 1, the docking structure 24 is a vertically loaded receptacle into which the portable ultrasound unit 12 may be inserted as shown by arrow 25. This is, however, merely one suitable arrangement for docking structure 24. The docking structure 24 may have a different configuration if desired. Another example of connecting and locking the portable ultrasound unit 12 to a horizontal surface of the docking cart is described below.

The docking structure 24 may be used to physically secure the portable unit 12 to the cart 14. Electrical connections between the portable ultrasound unit 12 and the docking cart 14 may also be made to allow information to be shared between the cart 14 and the portable unit 12.

The cart 14 is preferably substantially larger than the portable unit 12. For example, the cart 14 may be large enough to be pushed about on its wheels 26 by a standing user, without requiring that the user stoop or bend over excessively. Smaller or larger carts 14 may be provided if desired. Because the cart 14 has wheels 26, the weight of the cart 14 may be considerably greater than that of the portable unit 12. Each wheel 26 may be a swivel wheel that can be independently locked to prevent swivel movement, allowing the cart to be pushed down a corridor in a straight line. Wheels can also be locked to prevent rotation (e.g., to prevent the cart 14 from being stolen). The rotational or swivel motion of the wheels 26 may, if desired, be locked in unison (e.g., using a system of cables to mechanically actuated locks 27 together or by using electromagnetically actuated locks 27). The cart 14 may have any suitable number of wheels 26 (e.g., 3-8 wheels).

Because the cart 14 is larger than the portable unit 12 and may weigh more than the portable unit 12, the cart 14 may have features that can be used to supplement the capabilities of the portable unit 12. When the portable unit is mounted to the docking cart, the docking cart in effect transforms the portable unit into a cart-based system with enhanced features and functionality. These enhanced capabilities may include improved ergonomics, ease of use, a larger display format, external communications connectivity, supplemental transducer ports, increased data processing capabilities, and the like.

The cart 14 may have one or more supplemental displays such as a monitor 28 that may be used to enhance or replace the display capabilities of the portable ultrasound unit 12. The docking cart 14 may also have a user interface 134 and a processor 32 that can be used to supplement or replace the user interface and processing capabilities of the portable unit 12. For example, the user interface 134 may include a full-size keyboard for data entry, which may be easier to use than the data entry arrangement of the portable unit 12. As another example, the processor 32 may have more storage and greater or more flexible processing capabilities than the processor circuitry in the portable unit 12.

The enhanced processing capabilities of the cart 14 may be used to provide features that would otherwise be difficult or impossible to implement using only the portable unit 12. For example, the cart processor may be used to provide three-dimensional image rendering capabilities that are beyond the processing capabilities of the portable ultrasound unit operating alone. The cart's processor may also be used to implement powerful data processing packages. Large databases of ultrasound images or other patient data or reference-type medical data may be maintained by the cart's processor and associated storage devices. The cart's processor may help to coordinate access to network-based resources (e.g., medical data maintained on a hospital network).

A user may download patient data such as ultrasound image data or other data from the portable unit 12 to the cart 14. The downloaded data may be stored on the cart (e.g., in an image database or archive implemented using a hard drive) or may be stored on a network connected to the cart. The cart processor may be used to compare recently gathered patient images from the portable unit with historical images of the same patient or with other patient images that are maintained in the cart's database.

The images may be obtained from the portable unit in real time while the portable unit is docked or may be downloaded from the unit at some time after the images are acquired. By using the cart's processor to make comparisons between a patient's current images and that patient's archived images, a clinician can track changes in the patient's medical condition and thereby detect trends in the patient's condition. The clinician may also use the cart processor and imaging database capabilities of the cart to compare a patient's images to images of other patients or standard images in the image database.

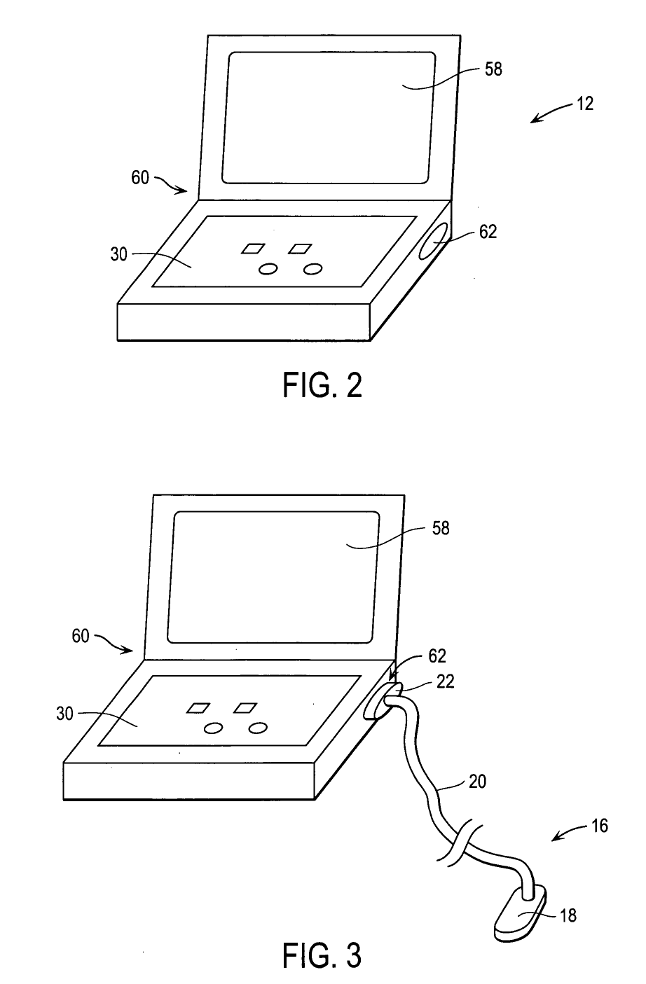

An illustrative portable ultrasound unit 12 is shown in FIG. 2. The portable unit 12 may have a built-in flat panel display screen 58 (e.g., a color LCD display). This display, which may measure about 5-17 inches diagonally, may be used to display ultrasound images and other information when the portable unit 12 is in operation. A hinge 60 may be used to allow the upper portion of portable unit 12 to fold down over the portable unit's user interface 30 when the portable unit 12 is not being used. The user interface 30 may include a track ball, joystick, touch pad or other pointing device, and buttons, knobs, keys, sliders, LEDs, speakers, microphones, and other suitable user interface equipment. Only a subset of such user interface devices are typically used on the portable unit 12, due to space and weight considerations. For example, the sliders can be omitted to save space.

The portable ultrasound unit 12 may have one or more transducer ports such as the transducer port 62. As shown in FIG. 3, an ultrasound transducer 16 may be attached to the portable ultrasound unit 12 by using the connector 22 to attach the cable 20 and the scanner head 18 to the port 62. Different transducers 16 may be attached to the portable unit 12 as needed depending on the ultrasound imaging task to be performed.

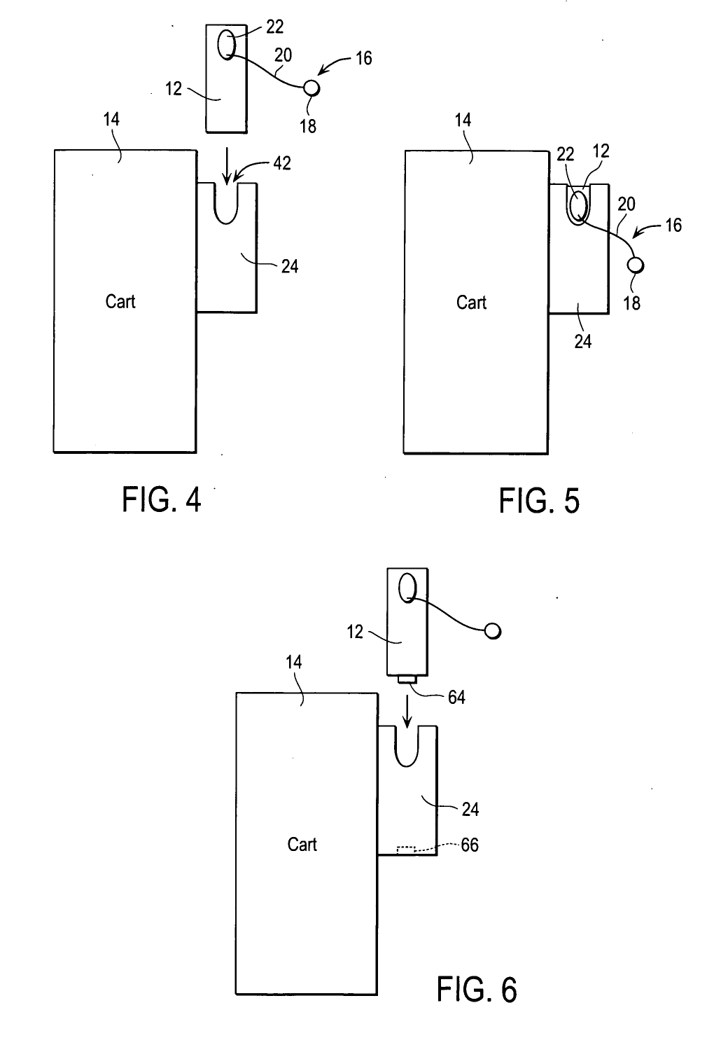

As shown in FIGS. 4 and 5, the docking structure 24 with which the portable ultrasound unit 12 is attached to the docking cart 14 may have a portion 42 that allows the connector 22 and the cable 20 of the transducer 16 to remain attached to the portable unit 12 even as the portable unit 12 is mated with the cart 14. This type of arrangement may be advantageous because it allows a user to continue using the same transducer that is attached to the portable unit 12 without interruption, even as the user transitions from using the user interface, display and other capabilities of the portable unit 12 to using the corresponding capabilities of the docking cart 14.

If desired, the portable ultrasound unit 12 and the docking cart 14 may have matching electrical connectors 64 and 66, as shown in FIG. 6. The connectors 64 and 66 may allow power and signals to be exchanged between the portable unit 12 and the docking cart 14. For example, ultrasound image data may be provided to the docking cart 14 from the portable unit 12 and power may be provided from the docking cart 14 to the portable unit 12 using the connectors 64 and 66. The connectors 64 and 66 may be provided using one connector or multiple connectors. When transferring ultrasound imaging data that is still in “channel” form, the connectors may include numerous parallel electrical connectors (differential and single-ended) for transmitted data to the cart 14 corresponding to each of the scanner array elements (channels) in the transducer head 18. The communications functions provided by the connectors 64 and 66 and their associated communications circuitry may also be provided using optical communications or RF communications arrangements.

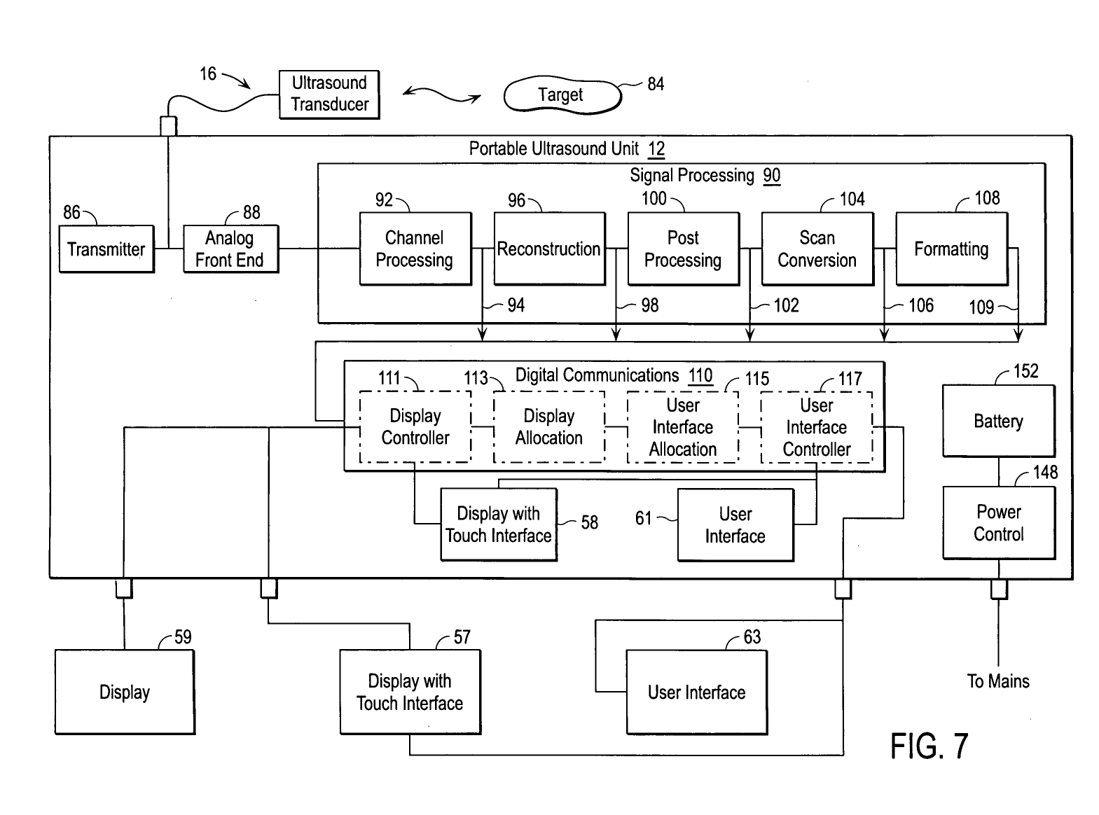

FIG. 7 is a schematic block diagram of an illustrative portable ultrasound unit 12 in accordance with an embodiment of the present invention. The ultrasound transducer 16 may be used to gather ultrasound images of a medical patient or other suitable image target 84. The portable ultrasound unit 12 has electronic circuitry for generating ultrasonic acoustic waves that are launched into the target 84 and has electronic circuitry for gathering and analyzing reflected acoustic waves to form corresponding ultrasound images. A transmitter 86, which may be a high-voltage transmitter, generates drive signals for the piezoelectric scanner elements in the ultrasound transducer 16. The resulting acoustic waves are reflected from the structure of the target 84. The scanner elements of the transducer 16 convert the reflected acoustic waves into electrical signals, which are processed by input circuitry in the portable unit 12.

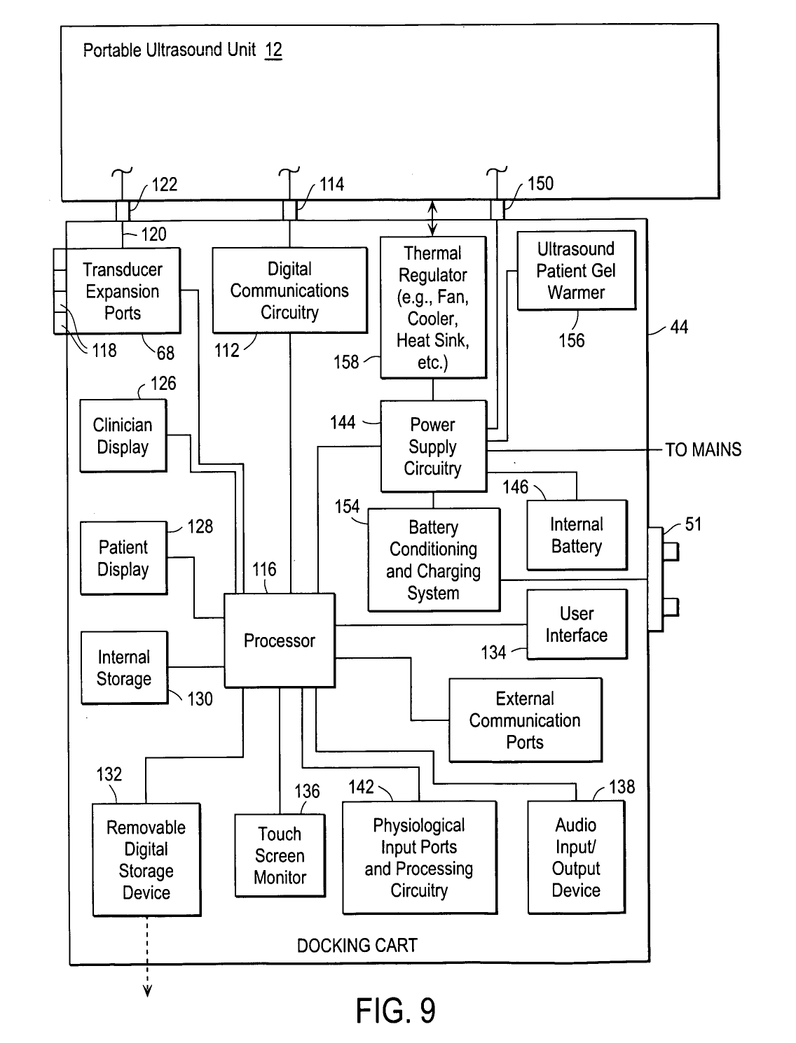

The input circuitry in the portable ultrasound unit 12 may include an analog front end 88 and other signal processing electronics 90. The circuitry of the analog front end 88 helps to condition the analog signals from the transducer 16 prior to digitization of these signals by the signal processing electronics 90. The transducer 16, which may either be directly connected to the portable unit 12 or which may be connected to the unit 12 through the docking cart's expansion ports (68 in FIG. 9), may have numerous (e.g., 100 or more) individual scanner elements, each of which generates a signal on a separate “channel.” Accordingly, the analog front end 88 may have circuitry that processes the analog input signals for each channel in parallel. The analog front end circuitry may include amplifier circuitry for amplifying signals detected by the transducer and may include analog filter circuitry for filtering out unwanted signals (e.g., based on their frequency). The conditioned analog signals from the analog front end 88 may be converted to digital signals by the digitization and channel processing circuitry 92.

The digitization and channel processing circuitry 92 may have analog-to-digital converters, buffer circuitry, and processing circuitry that digitize each channel of data in parallel, resulting in a total digital data throughput of about 10-1000 Gbps (or other suitable rate). The digitization and channel processing circuitry 92 may enhance the signal-to-noise ratio of the ultrasound image data by performing channel-domain processing tasks such as deconvolving coded signals to filter out unwanted signals. Following processing by the digitization and channel processing block 92, the ultrasound image signals may be provided at the output 94 as “channel data,” so-called because the data at this stage is still available on individual channels, each corresponding to a respective transducer array piezoelectric element.

The channel data must be processed further before a displayable image is created. An image reconstruction block 96 of the signal processing electronics 90 may be used to perform image reconstruction tasks (also called “beam formation tasks”). The resulting data signals at the output 98 may be referred to as “RF data” (data at a processing point after beam-formation, but prior to sampling and detection). The RF data at the output 98 still has both amplitude and phase information.

Further processing of the image data may be carried out using the post-processing portion 100 of the processing electronics 90. The resulting image data provided at the output 102 (called “detected data”) contains amplitude information, but no longer contains independent phase information.

The “detected data” image data may be processed further by the scan conversion portion 104 of the processing electronics 90 to produce “scan-converted data” at the output 106. The detected data processing performed by the scan conversion portion 104 may involve the use of acoustic-domain processing techniques that are based on a knowledge of the physical geometry of the transducer 16. Scan-converted data may be displayed as an ultrasound image on a display such as the portable ultrasound unit display 58 or external display 57 or 59 (which may be the display 126 or 128 of the cart in FIG. 9).

The scan-converted data produced at the output 106 of scan-conversion electronics 104 may be processed by formatting electronics 108 to produce corresponding “formatted image data” at the output 109. The formatted image data may be in a format suitable for viewing on a display such as the display 58 or 57 or 59. During formatting with the formatting electronics 108, content such as text or graphic overlays (e.g., annotations such as physician-entered annotations, time/date stamps, etc.) may be merged with the image to be displayed.

The digital image data from the signal processing electronics 90 of portable ultrasound unit 12 may be displayed in the portable ultrasound unit display 58 or external displays 57 or 59, or provided to the docking cart 14 in a number of different forms. As shown in FIG. 7, the portable ultrasound unit 12 may have digital communications circuitry 110 for supporting communications with various displays 57, 58, 59 and with the cart 14, as well as a portable ultrasound unit user interface 61 and an external user interface 63. The external displays 57, 59 and the external user interface 63 are optional. The portable unit 12 further includes an internal battery 152 and power control circuitry 148.

In the embodiment shown in FIG. 7, the digital communications circuitry 110 includes a display controller 111, a display allocation module 113, a user interface allocation module 115, and a user interface controller 117. The display allocation module 113 allocates display functionalities for a display with UI capability. The display controller 111 controls what display information or data goes to which display to perform the display functionalities. The user interface allocation module 115 allocates the user interface functionalities among the user interfaces. The user interface controller 117 controls what functionalities are performed by which user interface, for instance, by assigning a mapping of user interfaces and corresponding user interface functionalities.

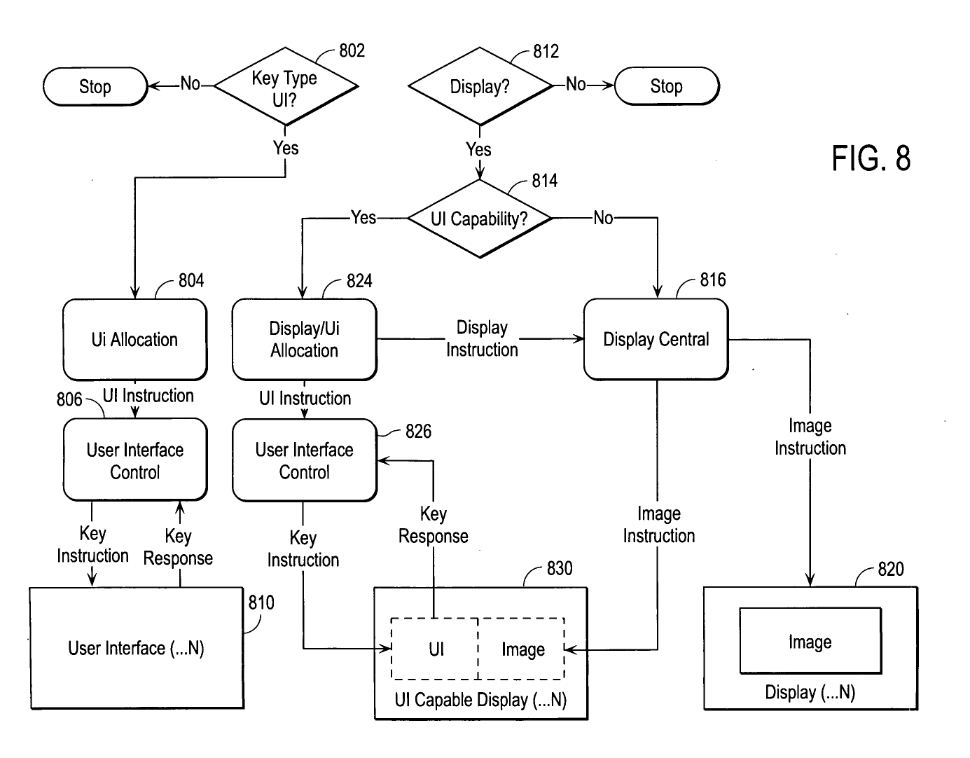

FIG. 8 is a flow diagram illustrating allocation and control of user interfaces and displays in the portable ultrasound unit according to an embodiment of the present invention. When the portable ultrasound unit 12 is powered up, the digital communications circuitry 110 obtains information regarding the available user interfaces and displays. Block 802 determines whether a particular resource is a key type user interface. If so, allocation of user interface (UI) functionalities for the key type user interface takes place in block 804. The UI allocation may be preprogrammed according to any desired criteria or scheme depending on what user interfaces are available to the portable unit 12. User interface instruction is generated in block 804 and passed to block 806 for user interface control of the specific user interface among the available user interfaces 810 based on the allocated UI functionalities. Key instruction is provided to the user interface being controlled, and key response is returned from the user interface to provide UI input to the portable unit 12.

The term “key type” user interface is used to distinguish it from a display-driven user interface (e.g., a touch screen display or graphical UI), and is not meant to limit the user interface to keys. The key type user interface may contain sliders (e.g., one or more sets of gain-depth-compensation sliders), knobs, buttons, keys (e.g., numeric keys, special functions keys, a full-size keyboard, etc.), and pointing devices (e.g., a mouse, trackball, joystick, keyboard-mounted pointing stick, touchpad, etc.). The keyboard of the user interface may be used for data entry (e.g., patient data entry) and image annotation. An advantage of providing a full-size keyboard either on the cart 14 or as an external keyboard connected to the portable unit 12 is that this allows easier data entry than the typically smaller user interface 61 of the portable unit 12 (see, e.g., FIG. 2). The pointing device and other controls may be used to navigate among various on-screen options that are shown on displays such as displays 57 and 59. Such on-screen options may, for example, allow the user to select which information is to be displayed on the cart's displays, to select which imaging modality is being used, to control settings, etc. Two sets of sliders may be used—a first one for adjusting the vertical gain/brightness of the display image and a second for adjusting the lateral gain/brightness of the display image. Special function keys may be used in the user interface to provide users with the ability to make single-key selections of options (e.g., to perform functions such as adjusting luminance curves, L/R invert, U/D invert, display format adjustment, sweep speed, acoustic output, Doppler gate size, etc.). These are merely illustrative user interface devices and ways in which such devices may be used to control the functions of the cart 14 and the portable unit 12. Any suitable user interface arrangement may be used to allow one or more users to interact with the docking cart 14 and the portable unit 12 as desired.

As seen in FIG. 8, block 812 determines whether a particular resource is a display. If so, block 814 determines whether the display has UI capability. If the display does not have UI capability, display control for the display takes place in block 816. Image instruction is generated and provided to display an image in the particular display among all available displays 820. If the display has UI capability, however, allocation of user interface (UI) functionalities for the display-driven user interface and allocation of display functionalities are performed, as seen in block 824. The allocation of UI and display functionalities for the UI capable display may be preprogrammed according to any desired criteria or scheme depending on the capabilities of the UI capable display. User interface instruction is generated in block 824 and passed to block 826 for user interface control of the UI aspect of the specific UI capable display among the available UI capable displays 830, such as touch screen displays. Display instruction is generated in block 824 and passed to block 816 for display control of the display aspect of the UI capable display. Key instruction is provided to the UI capable display being controlled, and key response is returned from the UI capable display to provide UI input to the portable unit 12. Image instruction is provided to the UI capable display to display image.

If the UI capable display 830 is sufficiently large, the screen may be split between UI and image. In some cases, the screen is not split but is switched between UI and image. For example, the UI display may overlay the image when it is enabled to receive user input, and disappear or hidden when it is desired to view the image.

As shown in FIG. 1, the cart 14 may include a user interface 134 and a processor 32 that can be used to supplement or replace the user interface and processing capabilities of the portable unit 12. FIG. 9 shows an example of such a cart 14. The cart 14 may have digital communications circuitry 112 for supporting communications with the portable unit 12. A connector 114 (partly implemented using a connector on the portable unit 12 and partly implemented using a connector on the cart 14) may be used to interconnect the circuitry 110 of the portable unit 12 and the circuitry 112 of the cart 14 (FIG. 7). The digital communications circuitry 110 and 112 may be used to support any suitable digital communications format. For example, data may be exchanged using serial protocols, parallel protocols, protocols for universal serial bus (USB) communications, IEEE 1394 (FireWire) communications, etc.

The image data supplied to the cart 14 by the portable ultrasound unit 12 may be provided in a relatively unprocessed form (e.g., as channel data at the output 94), in a relatively processed form (e.g., as formatted data at the output 109) as seen in FIG. 7. Data may also be transferred from the portable unit 12 to the cart 14 after an intermediate level of processing has been performed (e.g., as data at one or more of the outputs 98, 102, and 106). Providing image data to the cart 14 in a relatively unprocessed form may be advantageous when it is desired to retain a relatively large amount of flexibility for subsequent cart-based processing and when it is desired to avoid potentially irreversible losses of signal quality. Providing image data to the cart 14 in a relatively processed form may be advantageous when it is desired to reduce the processing burden on cart 14 and when this benefit outweighs the potential loss of flexibility in downstream signal processing that results from preprocessing the data.

The image data that is provided from the portable unit 12 to the cart 14 using the communications circuitry 110 and 112 may be provided in one format or only a few different formats (to simplify the processing circuitry in signal processing electronics 90). This image data may also be provided in many formats (e.g., all of the formats shown in FIG. 7).

If desired, the image data from the portable unit 12 may be provided to the cart 14 in the form of “channel data” at the output 94. Channel data includes signal samples gathered from each of the active piezoelectric elements in the transducer 16. The channel data is image data that has been digitized by the analog-to-digital converter circuitry of digitization and channel processing circuitry 92 of the processing electronics 90, but which has not yet undergone the beam formation process implemented by the image reconstruction electronics 96. An advantage of providing image data from the portable unit 12 to the cart 14 as channel data is that this allows the processing capabilities of the cart 14 to be used in handling the beam formation (image reconstruction) process.

Because the cart 14 may have a relatively powerful processor 116, the cart may, if desired, use such processing capabilities to perform more accurate or complete beam formation processing operations than would be possible using only the processing capabilities of the portable unit 12. Moreover, the beam formation operations of the cart may, if desired, be controlled by the user. For example, the cart may provide users with the ability to interact with on-screen options to make changes to the beam formation operation (e.g., through user-adjustable parameters). The user may, for example, make changes in the way the cart's processor handles velocity data, amplitude data, or other channel-based signal information.

If desired, the image data from the portable unit 12 may be provided to the cart 14 in the form of “RF data” at the output 98. RF image data is the data that has been through the image reconstruction process, but has not been sampled and detected. (The sampling and detection processes are performed by post-processing electronics 100.) RF image data still includes intact phase information. An advantage to providing image data to the cart 14 in the “RF data” format is that this allows the cart's processor to perform phase-related image-enhancement operations that are not possible once the phase information has been lost (as is the case with detected data). Substantially less bandwidth is required to transfer image data between the circuitry 110 and 112 in the form of RF data than in the form of channel data.

If desired, image data can be provided from the portable unit 12 to the cart 14 in the form of “detected data” at the output 102. An advantage of providing data as detected data rather than as RF data is that less processing is required to make the detected data displayable for the user. The detected data output stage of signal processing electronics 90 is the last stage at which an image for the display screen (e.g., the cart's display) can be generated in any desired native resolution without risk of compromising image quality (e.g., through resolution or image content losses). Detected data may, however, still be processed using acoustic-domain image processing techniques. If image data is provided from the portable unit 12 to the cart 14 at the “detected data” stage, rather than after processing the data further, the cart 14 can still be used to implement image processing tasks that are based on considerations of scanner (transducer head) geometry.

An additional reduction in the processing burdens on the cart 14 can be attained by providing image data from the portable unit 12 to the cart 14 in the form of “scan-converted data” at the output 106. Scan-converted data is data that has been converted from a format based on scanner geometry (detected data) to a user-display-oriented format. Image processing can still be performed on scan-converted data (if desired) using the amplitude information contained in the scan-converted data. For example, x-y filtering operations may be performed on the scan-converted data. The scan-converted data at the output 106 does not contain physician annotations or other overlay information. That information may be added by formatting electronics 108. An advantage of providing image data to the cart 14 in the form of scan-converted data is that the cart need only annotate the data (if desired) and convert the data to the proper screen format before displaying the data on one of the cart's displays. Because scan-converted data does not include annotations, this arrangement preserves the ability of the cart to display unannotated data.

Image data may also be provided from the portable unit 12 to the cart 14 in the form of “formatted image data” at the output 109. Formatted image data includes annotations (e.g., automatically generated annotations and annotations based on user input). Providing the image data to the cart as formatted image data reduces the image processing requirements of the cart to an extremely low level. Both scan-converted data and formatted image data have already been converted to a resolution that is specific to the screen format of the display 58 of the portable ultrasound unit 12, so this data is preferably converted (e.g., by the processor 116) to a format that is suitable for presentation on the display(s) of the cart 14. Formatted image data may be formatted (by either the portable unit 12 or subsequently by the cart 14) to accommodate standards such as DICOM, JPEG, TIFF, BMP, MPEG, or other suitable formats.

The processing capabilities of the cart 14 may be provided by the processor 116 and other components of the type shown in FIG. 9. The processor 116 may be based on one or more integrated circuits and other components. The processor 116 may, for example, be based on devices such as microcontrollers, microprocessors, personal computer boards, digital signal processors, programmable logic devices, application specific integrated circuits, memory devices, etc. In general, the capabilities of the processor 116 may be used to enhance the processing capabilities of the portable ultrasound unit 12, which are limited by size and weight considerations. The processor 116 may perform image processing tasks and may also serve as an embedded controller that controls the overall operation of the cart 14. Functions controlled by the processor 116 include coordinating input and output operations involving the user, ultrasound transducers, internal components, and peripheral devices.

In FIG. 9, the connectors 118 of the cart 14 that are used to attach the transducers 16 to the transducer expansion ports 68 are shown as being connected to the ports 68 from the exterior of the cart 14. To use a given transducer 16 that is connected to one of the transducer expansion ports 68, the processor 116 may activate multiplexer circuitry that switches a desired transducer 16 to the communications line 120.

The communications line 120 may be connected to the transmitter 86 of the portable unit 12 by the connector 122. High-voltage drive signals that are generated using the portable unit's transmitter 86 may be provided to a transducer 16 that is connected to one of the cart's expansion ports 68 via the connector 122 and line 120. Input signals from the same transducer may be routed through the expansion port 68 to the analog front end 88 of the portable unit 12 via the communications line 120, the connector 122, and a communications line from the connector 122 to the analog front end 88. The expansion port arrangement therefore allows the same high-voltage transmitter and analog front end (and some or all of the rest of the signal processing electronics 90 such as the digitization and channel processing circuitry 92) to be used to handle signals from both the transducer 16 that is connected to the connector 62 of the portable unit 12 and a transducer 16 that is connected to the expansion port.

The connector 122 (which may be partly implemented in the portable unit 12 and partly implemented in the cart 14) may be an electrical connector capable of passing numerous parallel channels of high-frequency signals having a large dynamic range (e.g., 160 dB or more). The connector 122 may, for example, be formed using the same types of electrical contacts and circuits used by the connector 62 (FIGS. 2 and 9) when connecting the main transducer 16 to the portable unit 12.

The expansion port capabilities of the cart 14 allow the larger size of the cart 14 to be used to overcome some of the size constraints faced by the portable ultrasound unit. With the expansion ports 68 of the cart 14, a user may attach multiple transducers 16 to the portable unit 12. The multiplexer circuitry that determines which of the transducers (main transducer 16 or one of the transducers connected via a given connector 118 attached to one of ports 68) is connected to the input and output electronics of the portable unit (e.g., the transmitter 86 and the analog-front-end 88) may be manually configured (e.g., through user interactions with the processor 116 through on-screen options) or may be automatically detected and configured (using mechanical or electronic detection of the presence or absence of a transducer at the ports 68).

The docking cart 14 may have one or more displays that supplement or replace the display capabilities of the portable ultrasound unit 12. For example, the docking cart 14 may have one or more clinician (user) displays 126. Such displays may be larger than would be desired on a portable device due to the size, weight and power constraints imposed by portability. More information may be displayed on the cart's displays than on the display of the portable unit 12. For example, additional information may be included on the cart display 126 (e.g., additional physician annotations, additional cart-generated annotations or overlay information, etc.). Additional image resolution and image content may be provided. The cart may, for example, display an image on a display 126 using the native resolution of that display (e.g., by using the cart's processor 116 to format the detected data from the portable unit into data in the desired native resolution).

The cart 14 may also include one or more patient displays such as a patient display 128. A patient display is intended to be viewable by a patient during use of the cart 14 and the portable unit 12 in performing ultrasound procedures. Patients cannot always see the monitors of traditional ultrasound units and are often not encouraged to do so because the monitors are awkwardly placed and because the images displayed on the monitors contain potentially disturbing physician annotations. The patient display 128 may be placed on an articulating arm or other support that makes it easy for the patient to view the image on the monitor without hindering the ability of the clinician to perform the ultrasound procedure. Moreover, some or all of the supplemental information (e.g., text and graphic overlays such as clinician annotations) that are displayed for the user (e.g., the physician or other clinician) on the clinician display 126 may be suppressed (not displayed) by the processor 116 before displaying the image for the patient. The images displayed on the patient display 128 will therefore be less likely to cause undue concern on the part of the patient viewing the images.

The docking cart 14 may have an internal storage 130. The internal storage may be formed using a hard drive, memory circuits (e.g., flash, RAM, ROM, EPROM, EEPROM), or any other suitable memory or storage device. The storage 130 may be used to store patient record data and image data (including stills and moving images) from the portable unit 12.

The docking cart 14 may also have removable storage. The cart 14 may, for example, have one or more removable storage devices 132 such as magneto-optic drives, diskette drives, compact flash slots or other memory card readers, writable CD or DVD drives, tape drives, etc. Removable storage media may be used when it is desired to archive a patient record or other information (e.g., an ultrasound video clip and associated physician annotations, etc.).

The docking cart 14 may have one or more user interface devices (shown generally as user interface 134 in FIG. 9). Displays such as the clinician display 126 and the patient display 128 may be used to display images and other information. If desired, one or more of the displays may be touch-sensitive, as shown by a touch-screen monitor 136. When the cart 14 has a touch-screen monitor such as the monitor 136, “soft menus” (e.g., user interface menus that processor 116 dynamically constructs out of on-screen options on the touch screen) can be used to provide a user of the cart 14 with user interface support. All or part of a given monitor may be provided with touch-screen capabilities.

An advantage of using a touch screen as a user interface for docking cart 14 is that this arrangement can help reduce clutter in the user console area. Ultrasound system operation can require many user adjustments. However, during certain modes of operation only a subset of the user controls are active. When the touch screen is used, inactive user control options can be hidden from view. Because inactive controls need not be displayed, they can either be hidden from view entirely (i.e., not displayed) or can be displayed in a way that indicates clearly to the user that those functions are currently inactive (e.g., by displaying the options with a reduced level of visibility on the screen relative to the options that remain active, by changing their color, etc.).

An audio input/output device 138, which is shown separately in FIG. 9, is a user interface device that may be used to present audio information to the user (e.g., the audio track associated with the spectral Doppler mode of operation of unit 12 that is picked up by a microphone associated with the portable unit 12 or a microphone associated with the cart 14).

The docking cart 14 may have one or more external communications ports 140. The communications circuitry of the ports 140 may be used to provide an interface between the processor 116 and the other components of the cart 14 and peripheral devices such as printers, plotters, recording devices (e.g., video recording devices such as VCRs or recordable DVD equipment), network equipment, telecommunications equipment, external displays, external storage devices, etc. Ports 140 may provide support for RS-232 signals and analog video.

The docking cart 14 may also have physiological input ports and processing circuitry 142. The input capabilities and processing capabilities of the physiological input ports and processing circuitry 142 may be used to gather (and process) information from external medical equipment.

The physiological input ports and processing circuitry 142 of the cart 14 can handle cardiac information, information from blood oxygen sensors, information from pulse sensors, information from respiration sensors, or any other suitable physiological equipment. In general, some or all of the processing of the raw sensor signals can be performed in the external equipment and corresponding digital information signals can be provided to processor 116 via port 142. If desired, the processing circuitry 142 may be used to handle signal conditioning and physiological data analysis tasks. Physiological data (or digital signals generated in response to processing the physiological data) may be shared with the portable ultrasound unit 12.

The docking cart 14 may draw power from an AC wall outlet (mains) or from an internal battery 146. The power supply circuitry 144 may be used to distribute power from the external supply or from the internal battery 146 to the components of cart 14. Power supply circuitry 144 may also supply power (from an external AC supply or from internal battery 146) to power control circuitry 148 of the portable ultrasound unit 12 via the connector 150. The power control circuitry 148 of the portable ultrasound unit 12 may be used to distribute power from the cart 14 or from the internal battery 152 to the components of the portable unit 12. The unit 12 may also use power from an AC source when not using power from the cart 14 or the battery 152. The power supply circuitry 144 may sense which type of AC source is connected to the cart 14 (e.g., 110 V, 60 Hz or 220 V, 50 Hz) and may adjust automatically to accommodate the characteristics of the AC source.

When the cart 14 is not connected to a source of AC power, the cart's internal battery 146 may be used to operate the cart's components and may (if desired) be used to supplement or replace the power supplied by the unit's battery 152. The cart's internal battery allows the cart to be readily transported from one room to another in a hospital or other establishment, without requiring that the user locate the cart near to an available wall outlet. The user can connect or disconnect the cart and AC power source at any time without interrupting the cart's operation.

The power supply circuitry 144 and power control circuitry 148 may be used to recharge the batteries 146 and 152 when AC power is available. The cart 14 may also have a battery conditioning and charging system 154 (and associated battery ports 156). The system 154 may be used to condition and charge the portable ultrasound unit's batteries (i.e., batteries such as internal battery 152 that have been removed from the unit 12). The battery charger of cart 14 may also be used to condition and charge other batteries (e.g., batteries for other portable medical equipment).

Ultrasound acoustic impedance matching gel is used to improve the efficiency of the acoustic impedance match between the face of transducer 16 and the target 84 (e.g., the tissue of the patient). The gel is typically applied directly to the skin of the patient. The docking cart 14 may have an ultrasound patient gel warmer 156 to warm the gel to a comfortable temperature before the gel is applied to the patient. The warmer 156 may warm the gel slightly above the ambient temperature of the room (e.g., to about 37° C. +/−5° C.). The gel warmer may be integrated into a cup-holder shaped structure (e.g., for holding plastic bottles of gel) or may have any other suitable shape. The gel warmer 156 may have a resistive or inductive heating element powered by power supply circuitry 144 or may use passive heating (e.g., the gel may be warmed by virtue of being located adjacent to a source of passive heating such as a warm portion of the cart's electronics, a heat sink, a fan outlet, etc.).

The portable ultrasound unit 12 is generally more exposed to the ambient atmosphere when used as a stand-alone unit than when the portable unit 12 is connected to the cart 14 and placed in a receptacle such as the holder 24 (FIG. 1). Exposure to surrounding air tends to cool the unit 12. The unit 12 may therefore experience a temperature rise when placed in a confined environment without supplemental cooling. Accordingly, the docking cart 14 may have a supplemental thermal regulator 158 that helps to control the temperature of portable ultrasound unit 12 when unit 12 is connected to cart 14.

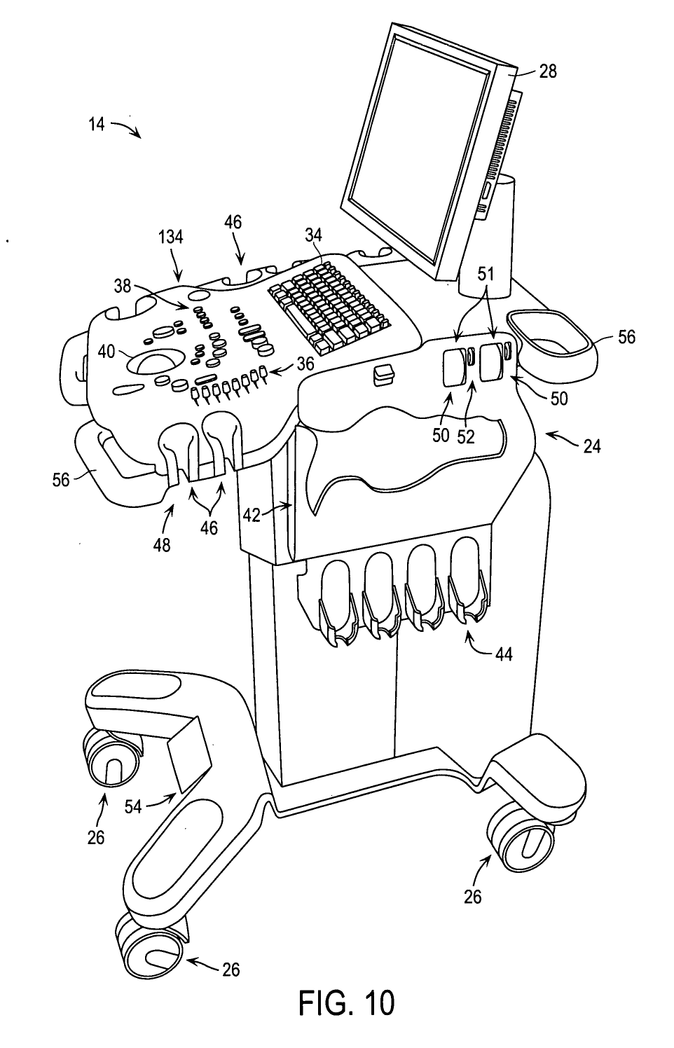

FIG. 10 is a perspective view of an illustrative docking cart in accordance with an embodiment of the present invention. The docking cart 14 has a large high-resolution monitor 28 such as a 19″ color LCD flat panel display. The monitor 28 may be any suitable size, but a larger monitor may be preferable in some working environments, because it presents a larger easier-to-view image for the user.

The docking cart 14 has a user interface 134 that includes a full-size keyboard 34, sliders 36 (e.g., for control of depth-gain compensation), various knobs and buttons 38, and a trackball 40. Other suitable user interface devices include touch pads, touch screens, voice recognition and audio equipment, a computer mouse, a joystick or other pointing device, etc.

The cart 14 has a docking structure or receptacle 24 into which the portable ultrasound unit 12 may be inserted. The receptacle 24 may have a cutout portion 42 that allows the transducer connector 22 and associated cable 20 of the transducer to protrude out of the portable ultrasound unit 12 while the unit 12 is attached to cart 14. The transducer head 18 of the transducer attached to the portable unit 12 and the transducer heads of additional transducers 16 may be placed in the transducer holders 44 or one of the additional holders 46 on the main control panel portion 48 of the docking cart. Holders such as the holders 46 may be used for any desired purpose such as for holding ultrasound gel, tissues, transducers or other medical instruments, etc.

The portable ultrasound unit 12 and other devices used by the user may be powered (at least some of the time) using batteries. The cart 14 may have integral battery charging ports 51. As shown in FIG. 2, batteries 50 may be charged and conditioned in these receptacles. If desired, the battery ports may each have an accompanying battery release mechanism activated by a button 52. The battery charging ports 51 may be used to recharge depleted batteries, may be used to recondition batteries in need of reconditioning, and may serve as a convenient storage location for charged fresh batteries.

The docking cart 14 may have wheels such as lockable swivel wheels 26 or any other suitable wheels or mechanisms for facilitating movement of docking cart 14. The wheels 26 may be locked by depressing a foot pedal 54 (which may be connected to one or more of wheels 26 by internal cabling) or by using other manually controlled or electronically controlled electromechanical actuators such as the locks 27 of FIG. 1. Other locking mechanisms may be used if desired. Handles such as front and rear handles 56 may be provided to allow the user to easily maneuver the cart.

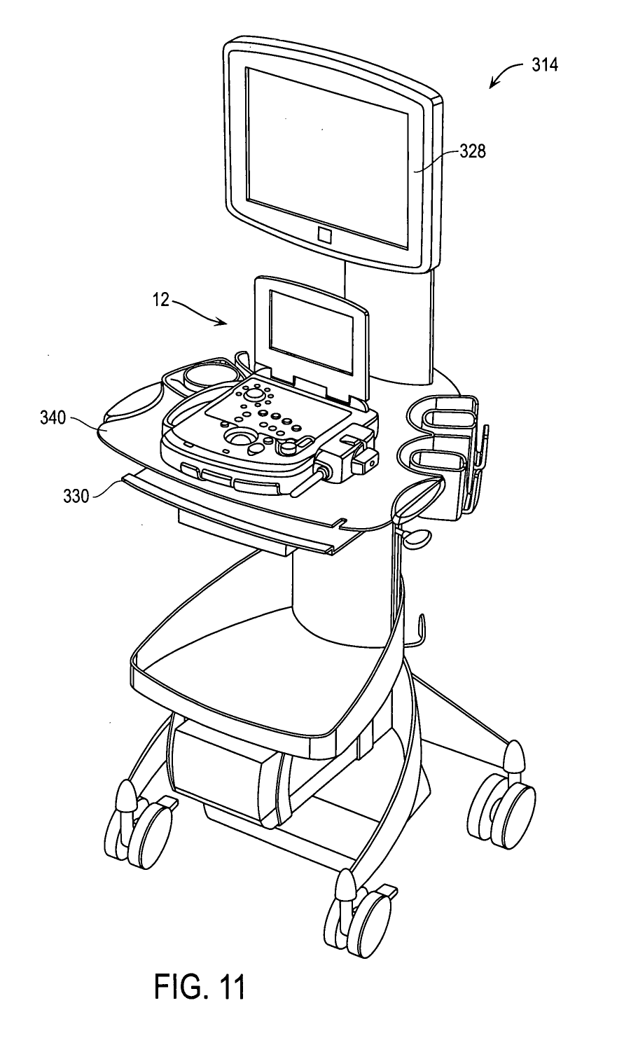

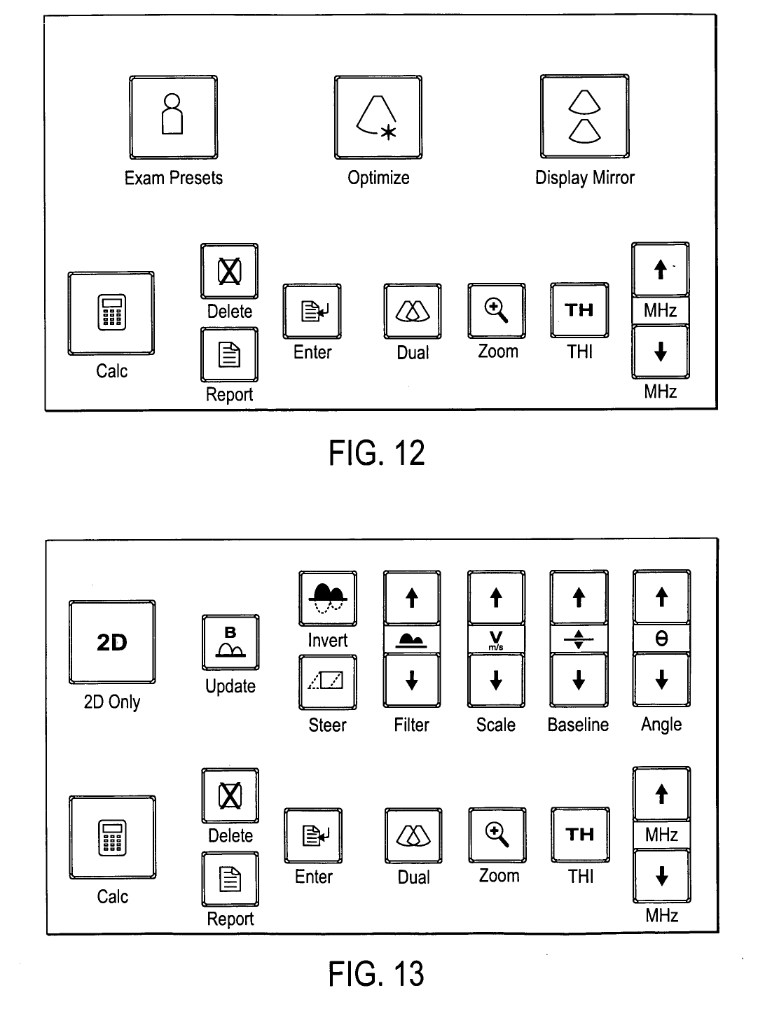

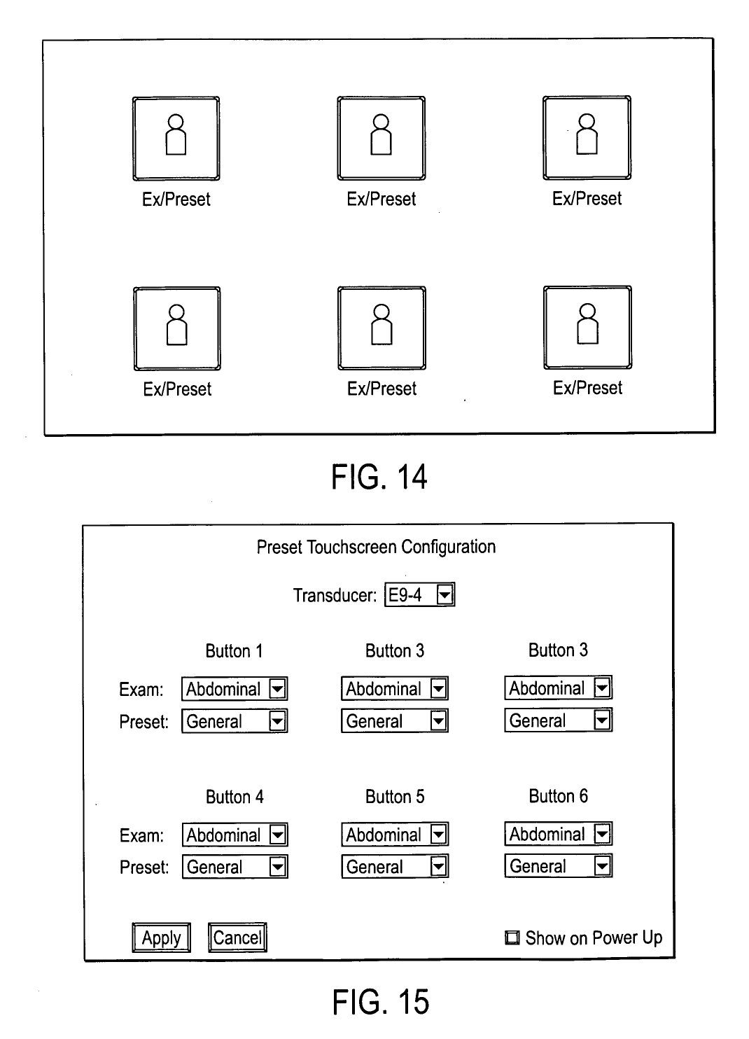

FIG. 11 is a perspective view of an illustrative docking cart in accordance with another embodiment of the present invention. The cart 314 of FIG. 11 has a simpler construction and contains fewer features than the cart 14 of FIG. 10. For example, while the cart 314 has a monitor 328, it does not provide the user interface 134 of the cart 14. Instead, the portable unit 12 provides the user interface capability. In such a system, the use of the display of the portable unit 12 as a touch screen interface or graphical UI, and/or operatively coupling a full-size keyboard to the portable unit, would be particularly advantageous. Such a keyboard as well as other user interface components may be connected to a USB port or the like of the portable unit, and be placed on a slidable tray 330 of the cart 314. The portable unit 12 typically includes various connection ports in the back for coupling to external devices and to the cart. Examples of touch screen interface displays include a touch screen main panel of FIG. 12, a Doppler mode panel of FIG. 13, a preset sub-panel of FIG. 14, and a preset touch screen configuration page of FIG. 15. The processor of the cart 314 in FIG. 11 can be selected to provide the fewer capabilities and need not be as powerful as the processor of the more complex cart 14 in FIG. 10.

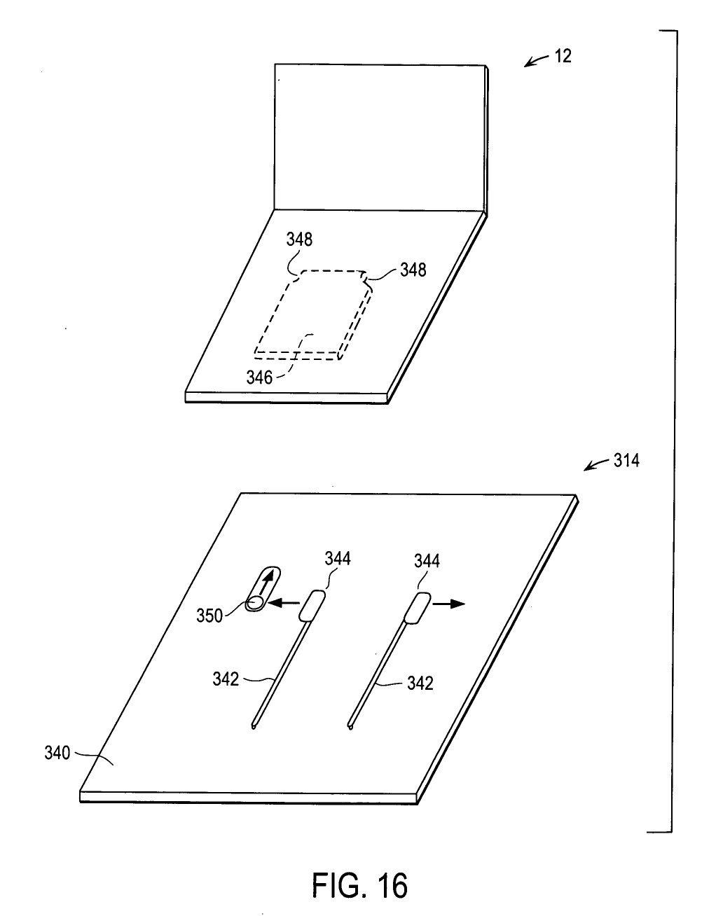

As shown in FIG. 11, the portable unit 12 is preferably supported on a generally horizontal surface 340 of the cart 314. FIG. 16 is a simplified view of a latch mechanism for locking the portable unit 12 to the surface 340 of the cart 314. A pair of rails 342 with locking tabs 344 are provided on the surface 340. The portable unit 12 includes a skid plate 346 with slots 348 to receive the locking tabs 344 as the skid plate 346 slides along the rails 342, thereby locking the skid plate 346 of the portable unit 12 to the rails 342 on the surface 340 of the cart 314. A latch 350 is connected to the locking tabs 344 and is movable to release the locking tabs 344 from the slots 348 of the portable unit 12 by pulling the tabs 344 away from one another (see arrows in FIG. 16), thereby allowing the portable unit 12 to be removed from the cart 314. The removal of the portable unit 12 does not require sliding the skid plate 346 along the rails 342 in the reverse direction. The portable unit 12 can be lifted up from the surface 340 when the latch 350 is moved to release the locking tabs 344.

As described above, the portable ultrasound unit 12 can operate on its own with the display screen 58, user interface 30, and, optionally, external user interface or similar components connected to the body of the portable unit 12. The display screen 58 is used to display ultrasound images, and may also be used as a touch screen display or graphical user interface as well.

The portable unit 12 may be connected to a docking cart to enhance the capabilities of the portable unit so that the portable unit's functionality may even rival or exceed the capabilities of traditional cart-based ultrasound equipment. Two examples of docking carts are described above. In the first cart 14 of FIG. 10, the docking cart 14 has a display monitor 28 and a full user interface 134. When the portable unit 12 is mounted to the first docking cart 14, the cart 14 may provide the full user interface and display capabilities, and there is no need to utilize the user interface and display capabilities of the portable unit 12. In the second cart 314 of FIG. 11, the docking cart 14 has a display monitor 328 but no user interface. When the portable unit 12 is mounted to the second docking cart 314, the cart 314 may provide superior display capabilities as well as additional data processing capabilities if needed. User interface functionalities are provided by the user interface on the body of the portable unit 12, external user interface components connected to the portable unit 12, and/or the portable unit display 58 functioning as a touch screen display or graphical user interface.

The portable unit 12 can be adapted to different stations or carts in a modular construction based on the application environment and capabilities of the different stations or carts. The ultrasound data obtained and processed in the portable unit can be provided to the particular station or cart for further processing and/or viewing. For example, ultrasound image data may be provided to the docking cart from the portable unit in the form of “channel data,” “RF data,”, “detected data,” scan-converted data,” or “formatted image data,” as discussed above. The respective processors of the portable unit and the particular cart communicate with one another and determine the data format and the respective user interface and display functions to be performed by the portable unit and the cart. In this way, a single portable unit can be used to collect and process ultrasound data, and be mounted to any suitable station or cart as a module to process and display the data as desired based on the application environment, programmed software, and the like.

It is to be understood that the above description is intended to be illustrative and not restrictive. Many embodiments will be apparent to those of skill in the art upon reviewing the above description. The scope of the invention should, therefore, be determined not with reference to the above description, but instead should be determined with reference to the appended claims along with their full scope of equivalents.

What is claimed is:

1. A portable ultrasound device comprising:
a portable housing;
a display control module configured to control a plurality of visual displays, at least one of the visual displays being selectively configurable to provide a user interface display on the visual display for user interface control, at least one of the visual displays being selectively configurable to view an ultrasound image; and
a plurality of user interfaces, at least one of the plurality of user interfaces being a separate user interface which is not integrally formed with the portable housing.

2. The portable ultrasound device of claim 1, wherein the display control module is configured to control visual displays having different sizes and/or resolutions.

3. The portable ultrasound device of claim 1, further comprising a portable ultrasound device visual display.

4. The portable ultrasound device of claim 3, wherein the portable ultrasound device visual display is selectively configurable to provide a user interface display on the portable ultrasound device visual display as a touch screen for user interface control of one or more of the user interfaces and selectively configurable to view an ultrasound image.

5. The portable ultrasound device of claim 1, wherein at least one of the user interfaces is an integral user interface which is integrally formed with the portable housing.

6. The portable ultrasound device of claim 1, further comprising a communication module configured to communicate with an auxiliary medical device for therapeutic application.

7. The portable ultrasound device of claim 6, wherein the communication module is configured to send control signal to and/or receive control signal from the auxiliary medical device.

8. The portable ultrasound device of claim 1, further comprising a security mechanism to lock the portable housing to a surface.

9. The portable ultrasound device of claim 1, further comprising a user interface allocation module configured to allocate user interface functionalities among the plurality of user interfaces.

10. The portable ultrasound device of claim 1, further comprising a visual display allocation module configured to allocate visual display functionalities among a plurality of visual displays.

11. An ultrasound system including the portable ultrasound device of claim 1 and a docking station, wherein the docking station comprises:
a docking station visual display which is configurable to view an ultrasound image.

12. The ultrasound system of claim 11,
wherein the portable ultrasound device comprises a transducer port configured to be coupled to a connector of an ultrasound transducer and a portable ultrasound device ultrasound processing circuitry that accepts first ultrasound image data from the transducer port and processes the first ultrasound image data to generate second ultrasound image data;
wherein the docking station comprises digital communications circuitry that supports communication between the docking station and the portable ultrasound device; and
wherein the docking station visual display is configured to display an ultrasound image based on the second ultrasound image data received from the portable ultrasound device through the digital communications circuitry.

13. The ultrasound system of claim 11,
wherein the docking station comprises digital communications circuitry that supports communication between the docking station and the portable ultrasound device;
wherein the docking station comprises a docking station ultrasound processing circuitry that processes the second ultrasound image data received by the docking station from the portable ultrasound device through the digital communications circuitry; and
wherein the docking station visual display is configured to display an ultrasound image based on processed data from the docking station ultrasound processing circuitry.

14. The ultrasound system of claim 11, wherein the docking station comprises a docking station user interface to receive user input of display instruction for the docking station visual display.

15. The ultrasound system of claim 11, wherein at least one of the user interfaces of the portable ultrasound device receives user input of display instruction for the docking station visual display.

16. The ultrasound system of claim 11, wherein the portable ultrasound device comprises a portable ultrasound device visual display, which is selectively configurable to provide a user interface display on the portable ultrasound device visual display, as a touch screen for user interface control to receive user input of display instruction for the docking station visual display.

17. An ultrasound system including a portable ultrasound unit and a docking cart,
wherein the portable ultrasound unit comprises a portable housing, a portable ultrasound unit visual display, and a plurality of user interfaces;
wherein the portable ultrasound unit visual display is selectively configurable to provide a user interface display on the visual display for user interface control of one of more of the user interfaces and selectively configurable to view an ultrasound image;
wherein the docking cart comprises a docking cart visual display which is configurable to view an ultrasound image; and
wherein the portable ultrasound unit further comprises a visual display allocation module configured to allocate visual display functionalities between the portable ultrasound unit visual display and the display cart visual display.

18. The ultrasound system of claim 17, wherein the portable user interface comprises a user interface allocation module configured to allocate user interface functionalities among the plurality of user interfaces.

19. The ultrasound system of claim 17, the plurality of user interfaces include at least one of:
a separate user interface which is not integrally formed with the portable housing; or
an integral user interface which is integrally formed with the portable housing.

20. The ultrasound system of claim 17, further comprising a security mechanism to lock the portable housing of the portable ultrasound unit to the docking cart.

21. The ultrasound system of claim 17,
wherein the portable ultrasound unit comprises a transducer port configured to be coupled to a connector of an ultrasound transducer and a portable ultrasound unit ultrasound processing circuitry that accepts first ultrasound image data from the transducer port and processes the first ultrasound image data to generate second ultrasound image data;
wherein the docking cart comprises digital communications circuitry that supports communication between the docking cart and the portable ultrasound unit; and
wherein the docking cart visual display is configured to display an ultrasound image based on the second ultrasound image data received from the portable ultrasound unit through the digital communications circuitry.

22. The ultrasound system of claim 17,
wherein the docking cart comprises digital communications circuitry that supports communication between the docking cart and the portable ultrasound unit;
wherein the docking cart comprises a docking cart ultrasound processing circuitry that processes the second ultrasound image data received by the docking cart from the portable ultrasound unit through the digital communications circuitry; and
wherein the docking cart visual display is configured to display an ultrasound image based on processed data from the docking cart ultrasound processing circuitry.

23. The ultrasound system of claim 17, wherein at least one of the user interfaces of the portable ultrasound unit receives user input of display instruction for the docking cart visual display.

24. The ultrasound system of claim 17, wherein the portable ultrasound unit visual display is selectively configurable to provide a user interface display on the portable ultrasound unit visual display, as a touch screen for user interface control to receive user input of display instruction for the docking cart visual display.

25. A portable ultrasound device comprising:
a portable housing;
a portable device processor;
a portable device visual display; and
at least one portable device user interface;
wherein the portable housing is configured to be mounted to any one of a plurality of docking stations;
wherein the portable device processor is configured to interface with a docking station processor of the docking station for allocating user interface functionalities and/or visual display functionalities between the portable ultrasound device and the docking station; and
wherein the visual display functionalities include displaying an ultrasound image.

26. The portable ultrasound device of claim 25, wherein the docking station has a docking station visual display, and wherein the visual display functionalities are allocated between the portable device visual display and the docking station visual display.

27. The portable ultrasound device of claim 26, wherein the portable device visual display is selectively configurable to provide a user interface display on the portable device visual display, as a touch screen for user interface control to receive user input of display instruction for the docking station visual display.

28. The portable ultrasound device of claim 26, wherein the visual display functionalities are fully allocated to the docking station visual display.

29. The portable ultrasound device of claim 25, wherein the docking station has at least one docking station user interface, and wherein the user interface functionalities are allocated between the at least one portable device user interface and the at least one docking station user interface.

30. The portable ultrasound device of claim 29, wherein the user interface functionalities are fully allocated to the at least one docking station user interface.
